(19) United States
(12) Patent Application Publication
(10) Pub. No.: US 2026/0256739 A1
(43) Pub. Date: Sep. 3, 2026

(54) THERAPEUTIC EFFECT OF MOLECULES DERIVED FROM PROBIOTIC MILK-BASED FERMENTATION MICROBIAL CONSORTIUM ("KEFIR") ON WOUNDS HEALING

(71) Applicant: B.G. Negev Technologies and Applications Ltd., at Ben-Gurion University, Be'er Sheva (IL)

(72) Inventors: Raz JELINEK, Reut (IL); Orit MALKA, Beer Sheva (IL)

(73) Assignee: B.G. Negev Technologies and Applications Ltd., at Ben-Gurion University, Be'er Sheva (IL)

(21) Appl. No.: 18/997,179

(22) PCT Filed: Jul. 31, 2023
(86) PCT No.: PCT/IL2023/050789

§ 371 Date: Jan. 21, 2025

Related U.S. Application Data
(60) Provisional application No. 63/393,926, filed on Jul. 31, 2022.

(51) Int. Cl. A61K 31/404; A61K 31/137; A61K 31/192; A61K 31/222; A61K 45/06; A61P 17/02; A61P 31/02

(52) U.S. Cl. CPC A61K 31/404; A61K 31/137; A61K 31/192; A61K 31/222; A61K 45/06; A61P 17/02; A61P 31/02

(57) ABSTRACT

The present invention is directed to a pharmaceutical topical composition comprising a Tryptophol derivative and a 4-Ethyl-Phenol derivative. Further provided are methods for using the composition.

### CROSS REFERENCE TO RELATED APPLICATIONS

This application claims the benefit of U.S. Ser. No. 63/393,926, filed on Jul. 31, 2022, which is incorporated in their entirety herein by reference.

### BACKGROUND OF THE INVENTION

Skin is the largest organ in the body and covers the body's entire external surface. It is made up of three layers, the epidermis, dermis, and the hypodermis, all three of which vary significantly in their anatomy and function. The skin's structure is made up of an intricate network which serves as the body's initial barrier against pathogens, UV light, and chemicals, and mechanical injury. It also regulates temperature and the amount of water released into the environment.

Skin disorders or diseases affect the skin and may result in a range of symptoms including inflammation, itching, burning, rashes, acne, and changes in pigmentation, among others.

Fissure is a common disease of the anus that causes massive suffering to those affected. In 2014, it was reported that 1100 patients were diagnosed in the United States for every 100,000 residents, and each year they are joined by another 235,000 diagnosed patients in the United States alone.

There is an ongoing need for therapies for skin pathologies with reduced side effects and induced efficacy.

### SUMMARY OF THE INVENTION

In some aspects, disclosed herein is a pharmaceutical topical composition comprising a Tryptophol derivative and a 4-Ethyl-Phenol derivative for use in the treatment of skin pathologies.

In some aspects, the Tryptophol derivative and a 4-Ethyl-Phenol derivative are in a ratio of 10:1-1:10 w/w ratio. In some further aspects, the Tryptophol derivative and a 4-Ethyl-Phenol derivative are in a ratio of 2:1-1:2 w/w ratio.

In some aspects, the Tryptophol derivative is Tryptophol acetate.

In some aspects, the 4-Ethyl-Phenol derivative is selected from the group consisting of: Tyrosol acetate, dopamine HCL, and caffeic acid.

In some aspects, the skin pathology comprises wounds, burns, burns after radiation in cancer patients, surgical wounds, wounds contaminated with bacteria, wounds contaminated with parasites, diabetic wounds, fusion of skin grafts, acne, eczema rashes, fissures, atopic dermatitis, allergy rashes, gout, joint pains, sprains, impetigo, leishmania, hemorrhoid, pruritus as a result of irritated skin, subcutaneous hemorrhages as a result of injuries or any combination thereof.

In some aspects, the skin pathology is in the scalp, back, hands, legs, face, front upper body area, backside, rectum, vagina, or any combination thereof.

In some aspects, the pharmaceutical topical composition comprises comprising 1250-2500 μM Tryptophol acetate and 1250-2500 μM Tyrosol acetate.

In some aspects, the composition is in the form of a solution, an ointment, a cream, a gel, a paste, a powder, a suspension, a lotion, a foam, a mousse, an aerosol, a spray, a microsphere, a microemulsion, a nanoemulsion, a nanosuspension, a dermal stick, a roll-on, a pump, a bandage, a patch, or a tape.

In some aspects, the pharmaceutical topical composition further comprising an active pharmaceutical ingredient.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is an illustration of the representative images of the wound in a treated mouse versus an untreated mouse in the days following the incision;

FIGS. 2A-C are an illustration of the antibacterial effect in an in vivo experiment of the examined cream. FIG. 2A is a schematic illustration of a mouse contaminated incision model. FIG. 2B is a representative pictures of a Staphylococcus aureus-contaminated skin wound on the back of the mouse on day 0, day 3 post-infection and day 10 post-infection with and without treatments. FIG. 2C is a bacterial culture taken from the contaminated wound showing the presence of S. aureus in the untreated mice compared to a clean culture of the treated mice on day 6;

FIG. 3 is an illustration of representative images of the sites on the back of the domestic swine (10 on each side) and treating the wounds with the ointment after they are created;

FIG. 4 is an illustration of the follow-up of healing of full-thickness excision wounds (12 mm diameter) on the back of a domestic swine using digital photography. Representative photos were taken on day 1, 3, 7, 10 and 14; and

FIG. 5 is a graph of the mean diameters of the non-treated wound and wounds treated with molecules cream, synthomycine 3% and negative control (vehicle) until day 10. Values are the averages of the results for tree measurements and error bars denote standard deviations. Student t-test calculated p-values of <0.05 were considered significant.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Composition

In some embodiments, disclosed herein is a pharmaceutical topical composition comprising a Tryptophol derivative and a 4-Ethyl-Phenol derivative for use in the treatment of skin pathologies.

A skilled artisan would understand the term “topical composition” as drug delivery as a route of administering drugs via the skin to provide topical therapeutic effects. As skin is one of the largest and most superficial organs in the human body, it is utilized to deliver various drugs. This system usually provides a local effect on certain positions of the body.

A skilled artisan would understand the term “skin pathologies” as conditions that affect the skin. These conditions may cause rashes, inflammation, itchiness or other skin changes. Some skin conditions may be genetic, while lifestyle factors may cause others. Skin disease treatment may include medications, creams or ointments, or lifestyle changes.

In some embodiments, the composition described herein comprises Tryptophol derivative and 4-Ethyl Phenol derivative in 1:15 to 15:1 w/w ratio. In some embodiments, the composition described herein comprises Tryptophol derivative and 4-Ethyl Phenol derivative in 1:14 to 14:1 w/w ratio. In some embodiments, the composition described herein comprises Tryptophol derivative and 4-Ethyl Phenol derivative in 1:13 to 13:1 w/w ratio. In some embodiments, the composition described herein comprises Tryptophol derivative and 4-Ethyl Phenol derivative in 1:12 to 12:1 w/w ratio. In some embodiments, the composition described herein comprises Tryptophol derivative and 4-Ethyl Phenol derivative in 1:11 to 11:1 w/w ratio. In some embodiments, the composition described herein comprises Tryptophol derivative and 4-Ethyl Phenol derivative in 1:10 to 10:1 w/w ratio. In some embodiments, the composition described herein comprises Tryptophol derivative and 4-Ethyl Phenol derivative in 1:9 to 9:1 w/w ratio. In some embodiments, the composition described herein comprises Tryptophol derivative and 4-Ethyl Phenol derivative in 1:8 to 8:1 w/w ratio. In some embodiments, the composition described herein comprises Tryptophol derivative and 4-Ethyl Phenol derivative in 1:7 to 7:1 w/w ratio. In some embodiments, the composition described herein comprises Tryptophol derivative and 4-Ethyl Phenol derivative in 1:6 to 6:1 w/w ratio. In some embodiments, the composition described herein comprises Tryptophol derivative and 4-Ethyl Phenol derivative in 1:5 to 5:1 w/w ratio. In some embodiments, the composition described herein comprises Tryptophol derivative and 4-Ethyl Phenol derivative in 1:4 to 4:1 w/w ratio. In some embodiments, the composition described herein comprises Tryptophol derivative and 4-Ethyl Phenol derivative in 1:3 to 3:1 w/w ratio. In some embodiments, the composition described herein comprises Tryptophol derivative and 4-Ethyl Phenol derivative in 1:2 to 2:1 w/w ratio. In some embodiments, the composition described herein comprises Tryptophol derivative and 4-Ethyl Phenol derivative in 1:1 to 1:1 w/w ratio. Each possibility represents a separate embodiment of the present invention.

In one embodiment, the composition described herein comprises Tryptophol derivative and 4-Ethyl Phenol derivative in 1:1 w/w ratio. In another embodiment, the composition described herein comprises Tryptophol derivative and 4-Ethyl Phenol derivative in 1:2 w/w ratio. In another embodiment, the composition described herein comprises Tryptophol derivative and 4-Ethyl Phenol derivative in 2:1 w/w ratio. In another embodiment, the composition described herein comprises Tryptophol derivative and 4-Ethyl Phenol derivative in 1:3 w/w ratio. In another embodiment, the composition described herein comprises Tryptophol derivative and 4-Ethyl Phenol derivative in 3:1 w/w ratio. In another embodiment, the composition described herein comprises Tryptophol derivative and 4-Ethyl Phenol derivative in 1:4 w/w ratio. In another embodiment, the composition described herein comprises Tryptophol derivative and 4-Ethyl Phenol derivative in 4:1 w/w ratio. In another embodiment, the composition described herein comprises Tryptophol derivative and 4-Ethyl Phenol derivative in 1:5 w/w ratio. In another embodiment, the composition described herein comprises Tryptophol derivative and 4-Ethyl Phenol derivative in 5:1 w/w ratio. In another embodiment, the composition described herein comprises Tryptophol derivative and 4-Ethyl Phenol derivative in 1:6 w/w ratio. In another embodiment, the composition described herein comprises Tryptophol derivative and 4-Ethyl Phenol derivative in 6:1 w/w ratio. In another embodiment, the composition described herein comprises Tryptophol derivative and 4-Ethyl Phenol derivative in 1:7 w/w ratio. In another embodiment, the composition described herein comprises Tryptophol derivative and 4-Ethyl Phenol derivative in 7:1 w/w ratio. In another embodiment, the composition described herein comprises Tryptophol derivative and 4-Ethyl Phenol derivative in 1:8 w/w ratio. In another embodiment, the composition described herein comprises Tryptophol derivative and 4-Ethyl Phenol derivative in 8:1 w/w ratio. In another embodiment, the composition described herein comprises Tryptophol derivative and 4-Ethyl Phenol derivative in 1:9 w/w ratio. In another embodiment, the composition described herein comprises Tryptophol derivative and 4-Ethyl Phenol derivative in 9:1 w/w ratio. In another embodiment, the composition described herein comprises Tryptophol derivative and 4-Ethyl Phenol derivative in 1:10 w/w ratio. In another embodiment, the composition described herein comprises Tryptophol derivative and 4-Ethyl Phenol derivative in 10:1 w/w ratio.

In one embodiment, the Tryptophol derivative is Tryptophol acetate.

In some embodiments, the 4-Ethyl-Phenol derivative is selected from the group consisting of: Tyrosol acetate, dopamine HCL, and caffeic acid. In one embodiment, the 4-Ethyl-Phenol derivative comprises Tyrosol acetate. In one embodiment, the 4-Ethyl-Phenol derivative comprises dopamine HCL. In one embodiment, the 4-Ethyl-Phenol derivative comprises caffeic acid.

In some embodiments, the composition is in the form of a solution, an ointment, a cream, a gel, a paste, a powder, a suspension, a lotion, a foam, a mousse, an aerosol, a spray, a microsphere, a microemulsion, a nanoemulsion, a nanosuspension, a dermal stick, a roll-on, a pump, a bandage, a patch, or a tape.

In one embodiment, the composition is in the form of a solution, In one embodiment, the composition is in the form of an ointment. In another embodiment, the composition is in the form of a cream. In another embodiment, the composition is in the form of a gel. In another embodiment, the composition is in the form of a paste. In another embodiment, the composition is in the form of a powder. In another embodiment, the composition is in the form of a suspension. In another embodiment, the composition is in the form of a lotion. In another embodiment, the composition is in the form of a foam. In another embodiment, the composition is in the form of a mousse. In another embodiment, the composition is in the form of an aerosol. In another embodiment, the composition is in the form of a spray. In another embodiment, the composition is in the form of a microsphere. In another embodiment, the composition is in the form of a microemulsion. In another embodiment, the composition is in the form of a nanoemulsion. In another embodiment, the composition is in the form of a nanosuspension. In another embodiment, the composition is in the form of a dermal stick. In another embodiment, the composition is in the form of a roll-on. In another embodiment, the composition is in the form of a pump. In another embodiment, the composition is in the form of a bandage. In another embodiment, the composition is in the form of a patch. In another embodiment, the composition is in the form of a tape.

In some embodiments, the composition further comprises an active pharmaceutical ingredient.

A skilled artisan would understand that an active pharmaceutical ingredient (API) is the component of an over the counter (OTC) or prescription medication that produces its intended health effects.

In one embodiment, the active pharmaceutical ingredient is a microorganism mixture. In one embodiment, the microorganism mixture comprises metabolic products of microorganisms, a matrix formed by the growth of microorganisms or a combination thereof.

In some embodiments, the microorganism mixture comprises Lactococcus lactis subsp, Lactis Lactobacillus kefiranofaciens ZW3, Lactobacillus, Leuconostoc mesenteroides, Kluyveromyces marxianus, Kazachstania turicensis or any combination thereof. In one embodiment, the microorganism mixture comprises Lactococcus lactis subsp. In another embodiment, the microorganism mixture comprises Lactis Lactobacillus kefiranofaciens ZW3. In another embodiment, the microorganism mixture comprises Lactobacillus. In another embodiment, the microorganism mixture comprises Leuconostoc mesenteroides. In another embodiment, the microorganism mixture comprises Kluyveromyces marxianus. In another embodiment, the microorganism mixture comprises Kazachstania turicensis.

In another embodiment, the active pharmaceutical ingredient comprises an anti-inflammatory agent, an anti-microbial agent, an anti-parasitic agent, an anti-allergy agent or any combination thereof. In another embodiment, the active pharmaceutical ingredient comprises an anti-inflammatory agent. In another embodiment, the active pharmaceutical ingredient comprises an anti-microbial agent. In another embodiment, the active pharmaceutical ingredient comprises an anti-parasitic agent. In another embodiment, the active pharmaceutical ingredient comprises an anti-allergy agent.

### Tryptophol Derivative and a 4-Ethyl-Phenol Derivative

In some embodiments, a Tryptophol derivative of the invention has the structure:

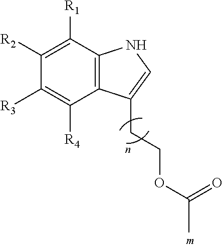

wherein “R1”, “R2”, “R3”, and “R4” are selected from the group consisting of: methyl (CH3), ethyl (CH3CH2), propyl (CH3CH2CH2) and butyl (CH3CH2CH2CH2);
“n” is a carbon chain comprising: one, two, three or four carbons; and
“m” is selected from the group consisting of: methyl (CH3), ethyl (CH3CH2), propyl (CH3CH2CH2) and butyl (CH3CH2CH2CH2).

In some embodiments, the Tryptophol derivative is Tryptophol acetate.

Tryptophol acetate is known in the art as having the structure:

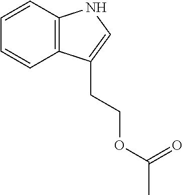

In some embodiments, a 4-Ethyl-Phenol derivative of the invention has the structure:

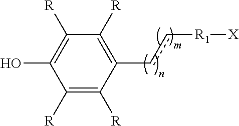

wherein each R is independently selected from the group consisting of: hydroxyl, hydrogen, methyl (CH₃), ethyl (CH₃CH₂), propyl (CH₃CH₂CH₂) and butyl (CH₃CH₂CH₂CH₂);
“n”, “m” are from 1 to 4,
R₁ comprises a heteroatom or is absent;
[symbol] represents a bond selected from the group consisting of: sp3 single C—C bond, sp2 double C—C bond, sp triple C—C bond; and
X is selected from the group consisting of: a carboxylic acid derivative, an alkyl, and hydrogen.

In some embodiments, a 4-Ethyl-Phenol derivative of the invention has the structure:

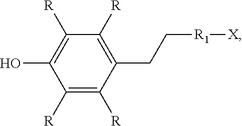

wherein R, R₁ and X are as described hereinabove.

In some embodiments, each R is independently selected from the group consisting of: hydroxyl, and hydrogen.

In some embodiments, R1 is selected from O, NH, and NH2.

In some embodiments, 4-Ethyl-Phenol derivative is a dopamine derivative represented by formula:

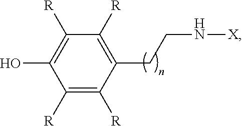

or by formula:

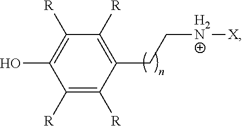

wherein R and X are as described hereinabove.

In some embodiments, a dopamine derivative is represented by formula:

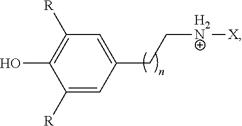

or by formula:

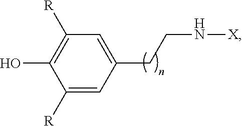

wherein R and X are as described hereinabove.

In some embodiments, X is hydrogen.

In some embodiments, 4-Ethyl-Phenol derivative is dopamine or a salt thereof.

In some embodiments, a 4-Ethyl-Phenol derivative of the invention has the structure:

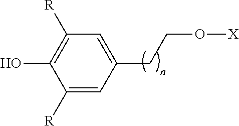

wherein: each R is independently selected from the group consisting of: hydroxyl, and hydrogen; and X is selected from the group consisting of: a carboxylic acid derivative, an alkyl, and hydrogen.

In some embodiments, X is

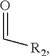

wherein R₂ is selected from the group consisting of: —OH, —SH, —NH₂, thioalkyl, oxyalkyl, aminoalkyl, hydrogen, alkyl, substituted alkyl.

In some embodiments, R₂ is hydrogen or an alkyl.

In some embodiments, R₂ is a C₁-C₅ alkyl.

In some embodiments, R₂ is hydrogen.

In some embodiments, the 4-Ethyl-Phenol is a derivative of Tyrosol acetate.

Tyrosol acetate is known in the art as having the structure:

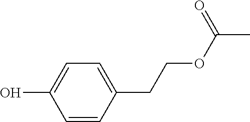

In some embodiments, a 4-Ethyl-Phenol derivative is a derivative of caffeic acid having the structure:

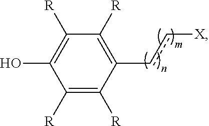

wherein each R is independently selected from the group consisting of: hydroxyl, hydrogen, methyl (CH3), ethyl (CH3CH2), propyl (CH3CH2CH2) and butyl (CH3CH2CH2CH2);
“n”, “m” are from 1 to 4,
[symbol] represents a bond selected from the group consisting of: sp3 single C—C bond, sp2 double C—C bond, sp triple C—C bond; and
X is selected from the group consisting of: a carboxylic acid derivative, an alkyl, and hydrogen.

In some embodiments, each R is independently selected from the group consisting of: hydroxyl, and hydrogen.

In some embodiments, [symbol] represents an unsaturated C—C bond. In some embodiments, [symbol] represents a double C—C bond.

In some embodiments, X is selected from the group consisting of: a carboxylic acid derivative, and hydrogen.

In some embodiments, the derivative of caffeic acid has the structure:

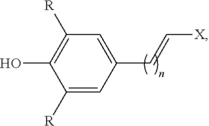

wherein R and X are as defined hereinabove.

In some embodiments, the derivative of caffeic acid has the structure:

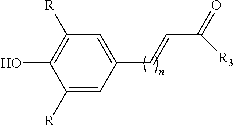

wherein R is as defined hereinabove, and R3 is selected from the group consisting of: hydrogen, —OH, —SH, —NH2, thioalkyl, oxyalkyl, aminoalkyl, hydrogen, alkyl, substituted alkyl.

In some embodiments, R is selected from the group consisting of: hydrogen, —OH, and alkyl.

In some embodiments, the derivative of caffeic acid has the structure:

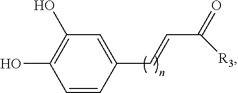

wherein R3 is as defined hereinabove.

As used herein, the term “carboxylic acid derivative” encompasses carboxy, amide, carbonyl, anhydride, carbonate ester, and carbamate.

As used herein, the term “derivative” encompasses any compound having antimicrobial activity that is generated from a similar compound by a chemical reaction, or any compound produced from another compound by substitution of one or more atoms. In some embodiments, the derivative comprises a structural analog.

In some embodiments, the compound of the invention is obtained by any chemical modification of Tryptophol or 4-Ethyl-Phenol, as long as it has antimicrobial activity. In some embodiments, Tryptophol or 4-Ethyl-Phenol are chemically modified by adding at least one chemical group selected from: acetylation, methylation, phosphorylation, amidation or others. In some embodiments, a chemical modification comprises substitution. In some embodiments, the modification comprises the addition of an acetate group to Tryptophol or 4-Ethyl-Phenol. In some embodiments, a Tryptophol acetate or Tyrosol acetate further comprises at least one chemical group as described above.

As used herein, the compound of the invention does not comprise Tryptophol or Tyrosol.

In some embodiments, the disclosed invention is directed to a composition comprising at least one molecule selected from: a Tryptophol derivative, and a 4-Ethyl-Phenol derivative, and at least one pharmaceutically acceptable carrier or diluent.

In some embodiments, the compound of the invention is chemically synthesized or biosynthesized. Methods of biosynthesis are well known within the art, and can include, but are not limited to: production in a cell culture or enzymatic cell-free production. In some embodiments, the compound of the invention is biosynthesized using a cell culture comprising Kluyveromyces marxianus. In some embodiments, the culture comprising K. marxianus is a mono- or polyculture. In some embodiments, the compound of the invention is biosynthesized by K. marxianus. In some embodiments, Tryptophol acetate or Tyrosol acetate are biosynthesized by K. marxianus according to the method of the present invention.

### Use

In some embodiments, the skin pathology comprises wounds, burns, burns after radiation in cancer patients, surgical wounds, wounds contaminated with bacteria, wounds contaminated with parasites, diabetic wounds, fusion of skin grafts, acne, eczema rashes, fissures, atopic dermatitis, allergy rashes, gout, joint pains, sprains, impetigo, leishmania, hemorrhoid, pruritus as a result of irritated skin, subcutaneous hemorrhages as a result of injuries or any combination thereof.

In one embodiment, skin pathology comprises wounds. In another embodiment, the skin pathology comprises burns. In another embodiment, the skin pathology comprises burns after radiation in cancer patients. In another embodiment, the skin pathology comprises surgical wounds. In another embodiment, the skin pathology comprises wounds contaminated with bacteria. In another embodiment, the skin pathology comprises wounds contaminated with parasites. In another embodiment, the skin pathology comprises diabetic wounds. In another embodiment, the skin pathology comprises fusion of skin grafts. In another embodiment, the skin pathology comprises acne. In another embodiment, the skin pathology comprises eczema rashes. In another embodiment, the skin pathology comprises fissures. In another embodiment, the skin pathology comprises atopic dermatitis. In another embodiment, the skin pathology comprises allergy rashes. In another embodiment, the skin pathology comprises gout. In another embodiment, the skin pathology comprises joint pains. In another embodiment, the skin pathology comprises sprains. In another embodiment, the skin pathology comprises impetigo. In another embodiment, the skin pathology comprises leishmania. In another embodiment, the skin pathology comprises hemorrhoid. In another embodiment, the skin pathology comprises pruritus as a result of irritated skin. In another embodiment, the skin pathology comprises subcutaneous hemorrhages as a result of injuries.

In some embodiments, the skin pathology is in the scalp, back, hands, legs, face, front upper body area, backside, rectum, vagina, or any combination thereof.

In one embodiment, the skin pathology is in the scalp. In another embodiment, the skin pathology is in the back. In another embodiment, the skin pathology is in the hands. In another embodiment, the skin pathology is in the legs. In another embodiment, the skin pathology is in the face. In another embodiment, the skin pathology is in the front upper body area. In another embodiment, the skin pathology is in the backside. In another embodiment, the skin pathology is in the rectum. In another embodiment, the skin pathology is in the vagina.

### Dosage and Administration

In some embodiments, the pharmaceutical topical composition comprises 1250-2500 μM Tryptophol acetate and 1250-2500 μM Tyrosol acetate.

In one embodiment, the pharmaceutical topical composition comprises 1250 μM Tryptophol acetate. In another embodiment, the pharmaceutical topical composition comprises 1500 μM Tryptophol acetate. In another embodiment, the pharmaceutical topical composition comprises 1750 μM Tryptophol acetate. In another embodiment, the pharmaceutical topical composition comprises 2000 μM Tryptophol acetate. In another embodiment, the pharmaceutical topical composition comprises 2250 μM Tryptophol acetate. In another embodiment, the pharmaceutical topical composition comprises 2500 μM Tryptophol acetate.

In one embodiment, the pharmaceutical topical composition comprises 1250 μM Tyrosol acetate. In another embodiment, the pharmaceutical topical composition comprises 1500 μM Tyrosol acetate. In another embodiment, the pharmaceutical topical composition comprises 1750 μM Tyrosol acetate. In another embodiment, the pharmaceutical topical composition comprises 2000 μM Tyrosol acetate. In another embodiment, the pharmaceutical topical composition comprises 2250 μM Tyrosol acetate. In another embodiment, the pharmaceutical topical composition comprises 2500 μM Tyrosol acetate.

In one embodiment, the pharmaceutical topical composition is administered once a day. In another embodiment, the pharmaceutical topical composition is administered twice a day. In another embodiment, the pharmaceutical topical composition is administered three times a day. In another embodiment, the pharmaceutical topical composition is administered four times a day. In another embodiment, the pharmaceutical topical composition is administered five times a day. In another embodiment, the pharmaceutical topical composition is administered six times a day.

In another embodiment, the pharmaceutical topical composition is administered every other day. In another embodiment, the pharmaceutical topical composition is administered twice a week. In another embodiment, the pharmaceutical topical composition is administered once a week.

In one embodiment, the pharmaceutical topical composition is administered for 3 days. In another the pharmaceutical topical composition is administered for 4 days. In another the pharmaceutical topical composition is administered for 5 days. In another the pharmaceutical topical composition is administered for 6 days. In another the pharmaceutical topical composition is administered for 7 days. In another the pharmaceutical topical composition is administered for 8 days. In another the pharmaceutical topical composition is administered for 9 days. In another the pharmaceutical topical composition is administered for 10 days. In another the pharmaceutical topical composition is administered for 20 days. In another the pharmaceutical topical composition is administered for 30 days.

In another the pharmaceutical topical composition is administered in chronic administration.

A skilled artisan would understand the meaning of “chronic administration” as administration over a long period of time, varying from weeks to months and years. Chronic administration can also mean treating a condition or disease that is persistent, constantly, recurring or otherwise long-lasting in its effects or a disease that comes with time, therefore the chronic administration can be periodically administration.

### EXAMPLES

### Example 1—In Vivo Experiment in Mice

The treatment was tested in two preliminary experiments on mice to test the safety and therapeutic effect of the molecules on acute wounds and acute wounds contaminated with Staphylococcus aureus bacterium.

In the experiments, a deep cut (1.5 cm long) was made using a scalpel or sharp scissors on the back of the mouse until the fascia layer was removed. The treatment was carried out in two ways—in the first experiment the molecules dissolved in water were dripped onto the wound and in the second experiment the cream with the molecules (600 μM 1:1 ratio) was applied to the wound. The treatments were carried out immediately after performing the incision and were given twice a day for 4 days.

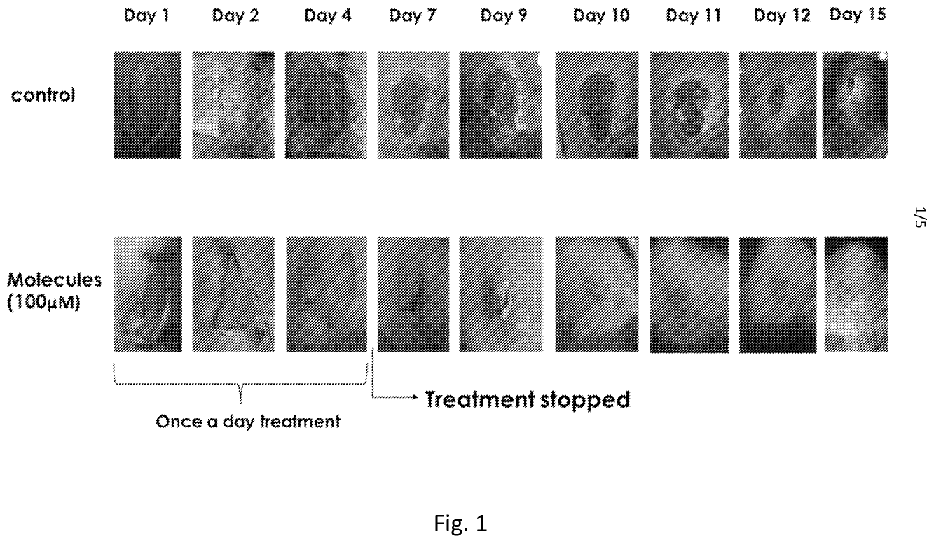

The sample results of the first experiment are shown in FIG. 1 and exhibit almost complete healing of the incision in the treated mouse in half the time it took to heal the incision in the untreated mouse. In addition, no infection was observed in the wounds of the treated mouse.

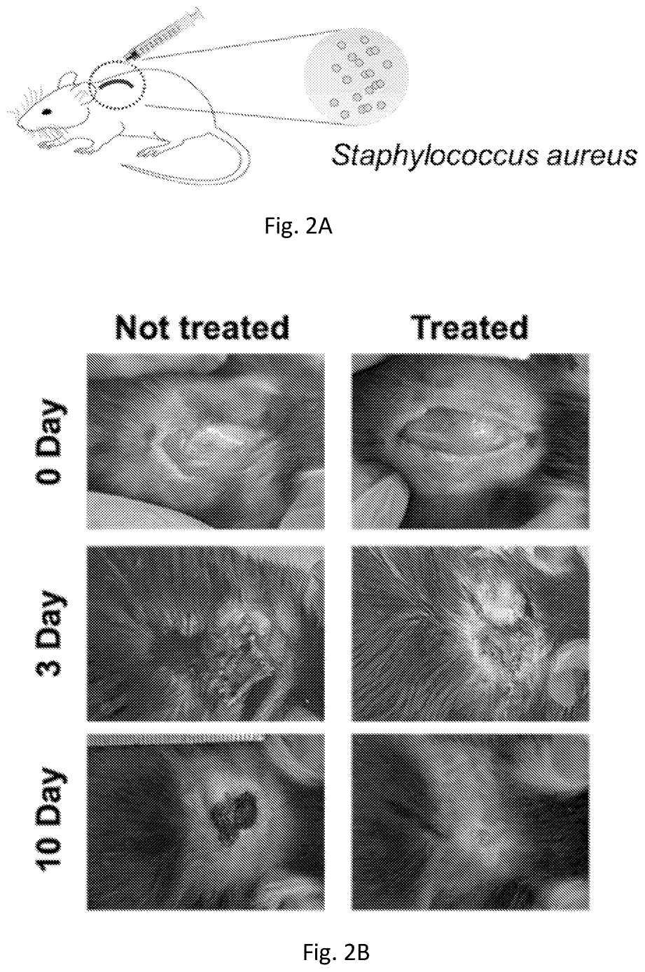

In the second experiment, the wounds were even contaminated by a culture of S. aureus bacterium to simulate acute infection situations in the skin when a culture was taken from the wound every day to examine the presence of bacteria (FIG. 2A). The results obtained showed that the incision was almost completely healed on the 10th day in the treated mouse (FIG. 2B). Moreover, on day 6, the culture test showed that there was no bacterial presence in the wound compared to the untreated mouse (FIG. 2C).

### Example 2—Preclinical Experiment in Porcine

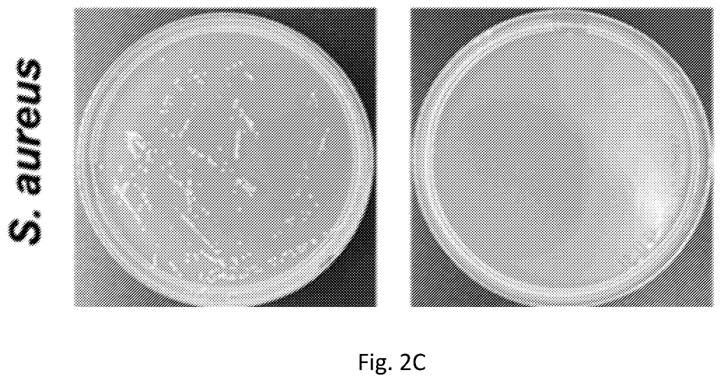

In the present experiment two domestic swine weighing ~20 kg each (simulating adult human skin in the best way) were used. 20 sites were drawn on the back of the pig (10 on each side; FIG. 3) and created wounds with the help of PUNCH BIOPSIES and immediately applying the ointment containing the active substance while examining different doses ratios between the molecules—Tp: tryptophol acetate; Ty: Tyrosol acetate. According to Table 1 below.

TABLE 1
different doses ratios between the molecules -
Tp: tryptophol acetate; Ty: Tyrosol acetate

| Group | Ointment dose |
|---|---|
| 1 | Tp:Ty 1:1 total concentration 2000 μM |
| 2 | Tp:Ty 3:7 total concentration 2000 μM |
| 3 | Tp:Ty 1:1 total concentration 1000 μM |
| 4 | Tp:Ty 3:7 total concentration 1000 μM |
| 5 | Synthomycine 3% as positive control |
| 6 | Negative control (vehicle) |

The above treatments were applied until day 7, twice a day and followed the wound healing process until the 14th day when the animals were sacrificed.

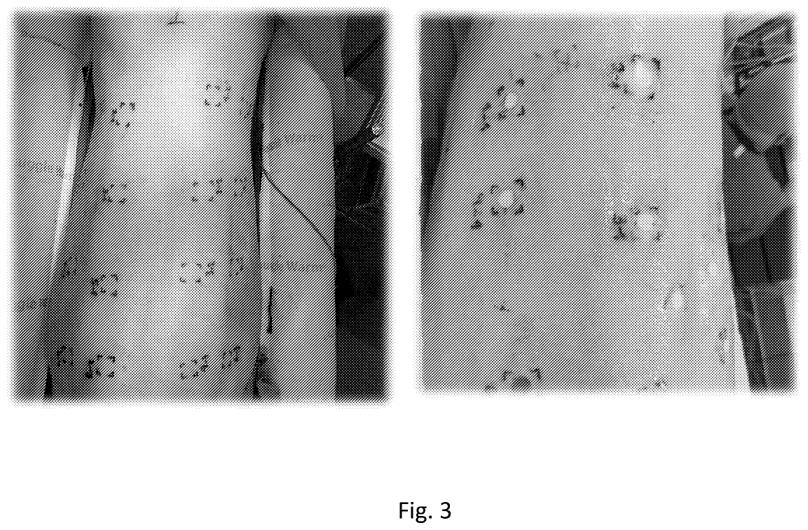

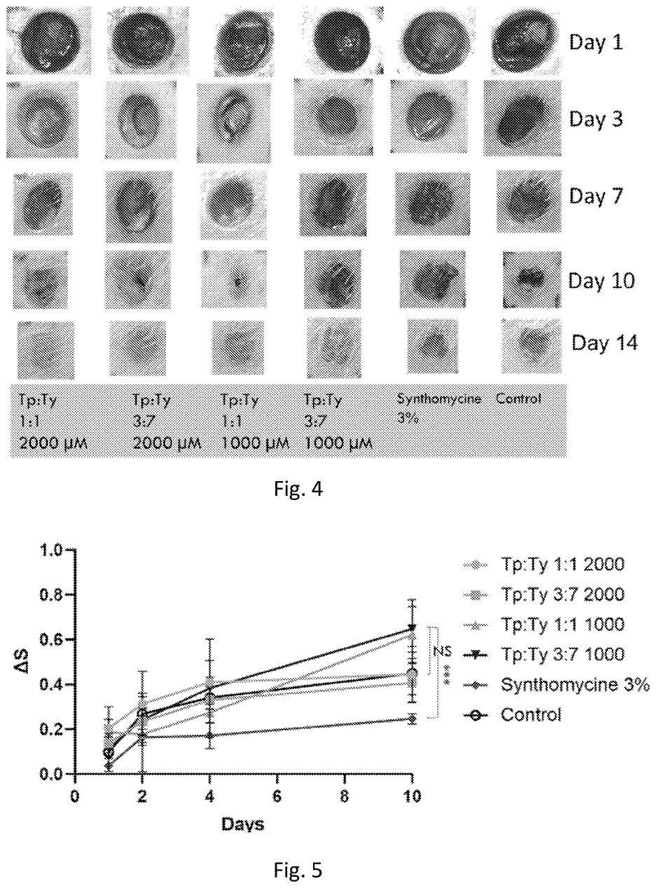

The results are presented in FIG. 3, FIG. 4 and FIG. 5.

The results indicate that on day 10, the wounds treated with Tp:Ty 1:1 2000 μM, Tp:Ty 3:7 2000 μM and Tp:Ty 1:1 1000 μM were almost healed relative to the negative control and antibiotic groups (FIG. 4). The changes in the area of the wounds were monitored during the experiment and measured on days 1, 2, 4, and 10 (FIG. 5). The observed decrease in the wound area of Tp:Ty 1:1 1000 μM and Tp:Ty 3:7 1000 μM treatments indicating faster healing that were significant compared to that obtained for the negative and positive control. Overall, when the treatment with the creams used in ratio of 1:1 and with total concentration of 1000 μM it was found to accelerate the healing process better than antibiotics (syntomycin 3%) or the vehicle.

### Example 3—a Unique Topical Mixture for the Treatment of Skin Lesions Based on Molecules Isolated from Probiotic Microorganisms in Fermented Milk

The aim of this example is to examine the topical ointment comprising tryptophol acetate and Tyrosol acetate in three different concentrations (of both molecules together)—1000, 2000 and 4000 μM for local treatment of patients with acute fissures.

The results of the trial are examined in two stages:

Step 1—The safety of using the ointment and maximum concentration will is assessed while examining the effect and side effects through telephone questionnaires and doctor examinations. A total of 12 patients with an acute crack in the anus are recruited at this stage. The patients are divided into 3 groups of 4 patients in each group and receive the ointment in three different concentrations. The goal is to examine the side effects and maximally tolerated dose.

Stage 2 is performed after the completion of Phase 1 and assuming no significant side effects are detected. At this stage, the initial effectiveness of the ointment is assessed by continuous monitoring of the patients who are treated with the ointment in addition to the standard treatment accepted in the medical protocol. The number of patients with anal fissure required for this stage is 51. Of these, 17 are in the control group and 34 in the intervention group (2:1 ratio).

A total of 63 patients are recruited, of whom 12 are for the first phase and 51 for the second phase.

### Methods—

Two groups are included in the experiment:
Group 1—Control Patients with an acute fissure in the anus who receive the recommended standard of care.
Group 2—Intervention of patients with an acute crack in the anus who receive the recommended standard treatment such as the control group and application of the tested topical mixture.

### Method of Treatment:

The participant is receiving a syringe with a 20 ml cream containing a mixture of the two molecules in a dose to be determined in step 1. The cream is manufactured in a pharmaceutical preparation by a GPP approved pharmacy (Good Production Practices) or GMP (Good Manufacturing Practices). This treatment is given in addition to the recommended treatment, which is the application of enterolin cream twice a day in both study groups. The time of ointments application is separated by at least one hour from each other.

The duration of treatment: Three times a day for 10 days.

The examination of statistical significance:

The sample size for phase 2 is calculated in the WINPEP software, according to an expected difference of 2 units in the degree of suffering and pain at the control point with a standard deviation of 2, a significance of 5%, and a power of 80%, with a ratio of 2:1 between the groups. The minimum sample size is determined to be 36 subjects, of which 24 are in the intervention group and 12 are in the control group (36 in total).

### Safety Analysis:

The side effects are recorded in an Excel table and monitored in real time, in addition, the side effects are summarized according to the relationship to the treatment and according to the severity of the phenomenon.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of treating skin pathologies comprising administering a pharmaceutical topical composition comprising a Tryptophol derivative and a 4-Ethyl-Phenol derivative to a subject in need thereof.

2. The method according to claim 1, wherein said Tryptophol derivative and a 4-Ethyl-Phenol derivative are in a ratio of 10:1-1:10 w/w ratio.

3. The method according to claim 2, wherein said Tryptophol derivative and a 4-Ethyl-Phenol derivative are in a ratio of 2:1-1:2 w/w ratio.

4. The method according to claim 1, wherein said Tryptophol derivative is Tryptophol acetate.

5. The method according to claim 1, wherein said 4-Ethyl-Phenol derivative is selected from the group consisting of: Tyrosol acetate, dopamine HCL, and caffeic acid.

6. The method according to claim 1, wherein said skin pathology comprises wounds, burns, burns after radiation in cancer patients, surgical wounds, wounds contaminated with bacteria, wounds contaminated with parasites, diabetic wounds, fusion of skin grafts, acne, eczema rashes, fissures, atopic dermatitis, allergy rashes, gout, joint pains, sprains, impetigo, leishmania, hemorrhoid, pruritus as a result of irritated skin, subcutaneous hemorrhages as a result of injuries or any combination thereof.

7. The method according to claim 1, wherein said skin pathology is in the scalp, back, hands, legs, face, front upper body area, backside, rectum, vagina, or any combination thereof.

8. The method according to claim 1, wherein said skin pathology comprises fissures.

9. The method according to claim 4 and 5, comprising 1250-2500 μM Tryptophol acetate and 1250-2500 μM Tyrosol acetate.

10. The method according to claim 1, wherein said composition is in the form of a solution, an ointment, a cream, a gel, a paste, a powder, a suspension, a lotion, a foam, a mousse, an aerosol, a spray, a microsphere, a microemulsion, a nanoemulsion, a nanosuspension, a dermal stick, a roll-on, a pump, a bandage, a patch, or a tape.

11. The method according to claim 10, wherein said composition is in the form of an ointment.

12. The method according to claim 10, wherein said composition is in the form of a cream.

13. The method according to claim 1, further comprising an active pharmaceutical ingredient.

14. The method according to claim 13, wherein said active pharmaceutical ingredient is a microorganism mixture.

15. The method according to claim 13, wherein said active pharmaceutical ingredient comprises an anti-inflammatory agent, an anti-microbial agent, an anti-parasitic agent, an anti-allergy agent or any combination thereof.
